(19) United States
(12) Patent Application Publication
(10) Pub. No.: US 2020/0046203 A1
(43) Pub. Date: Feb. 13, 2020

(54) MEDICAL SYSTEM

(71) Applicant: OLYMPUS CORPORATION, Tokyo (JP)

(72) Inventor: Takahiro KOMURO, Tokyo (JP)

(73) Assignee: OLYMPUS CORPORATION, Tokyo (JP)

(21) Appl. No.: 16/658,825

(22) Filed: Oct. 21, 2019

Related U.S. Application Data
(63) Continuation of application No. PCT/JP2017/016547, filed on Apr. 26, 2017.

(51) Int. Cl. A61B 1/00; A61B 1/005

(52) U.S. Cl. CPC A61B 1/00057; A61B 1/00135; A61B 1/0002; A61B 1/00006; A61B 1/00059; A61B 1/0057

(57) ABSTRACT

A medical system includes: an elongated medical device having flexibility; an elongated over-tube having flexibility and having a channel into which the medical device is inserted in a longitudinal direction; a movement mechanism including a slider attached to a proximal end of the medical device, the movement mechanism being configured to move the medical device in the longitudinal direction relative to the over-tube; a wire fixed to a distal end of the over-tube and extending parallel to the channel, the wire being supported in a movable manner only in the longitudinal direction relative to the over-tube; a sensor that detects a protruding length of the wire from the proximal end of the over-tube; and a controller that calculates a path length of the channel based on the protruding length of the wire detected by the sensor.

### CROSS-REFERENCE TO RELATED APPLICATIONS

This is a continuation of International Application PCT/JP2017/016547 which is hereby incorporated by reference herein in its entirety.

### TECHNICAL FIELD

The present invention relates to medical systems.

### BACKGROUND ART

A known medical system causes a roller to roll on a surface of an endoscope and measures the insertion length of the endoscope in accordance with the rotation amount of the roller when the endoscope is to be inserted into a channel in an over-tube (e.g., see Patent Literature 1).

When a medical device is to be inserted into the channel in the over-tube, it is sometimes desirable to limit the minimum value of the protruding amount of the medical device from the distal end of the over-tube. For example, if the medical device has joints at the distal end thereof, the minimum value of the protruding amount is limited to the protruding amount at a position where the joint at the extreme proximal end is not retracted into the channel, thereby preventing problems, such as an inability to perform appropriate operations after the medical device or the over-tube is damaged due to movement of the joints in a state where they are retracted into the channel.

### CITATION LIST

### Patent Literature

### {PTL 1}

The Publication of Japanese Patent No. 4348484

### SUMMARY OF INVENTION

### Technical Problem

However, in a case where channels are disposed at positions decentered from the center of the over-tube in the radial direction, there is a problem in that the path lengths of the channels change when the over-tube is bent. In other words, the path length of the channel located at the outer side of the bend increases, whereas the path length of the channel located at the inner side of the bend decreases.

Therefore, even if the medical device is inserted into a channel by an insertion length set in accordance with the path length of the channel in the over-tube in a straight state, the path length of the channel increases depending on the bent shape of the over-tube. This is problematic in that the actual protruding amount of the medical device from the distal end of the over-tube is smaller than the minimum value.

The present invention has been made in view of the circumstances described above, and an object thereof is to provide a medical system in which a medical device can be disposed at an appropriate position in accordance with a bent shape of an over-tube.

### Solution to Problem

An aspect of the present invention provides a medical system including: an elongated medical device having flexibility; an elongated over-tube having flexibility and having a channel into which the medical device is inserted in a longitudinal direction; a movement mechanism including a slider attached to a proximal end of the medical device, the movement mechanism being configured to move the medical device in the longitudinal direction relative to the over-tube; a wire fixed to a distal end of the over-tube and extending parallel to the channel, the wire being supported in a movable manner only in the longitudinal direction relative to the over-tube; a sensor that detects a protruding length of the wire from the proximal end of the over-tube; and a controller that calculates a path length of the channel based on the protruding length of the wire detected by the sensor.

### BRIEF DESCRIPTION OF DRAWINGS

FIG. 1 is an overall configuration diagram schematically illustrating a medical system according to an embodiment of the present invention.

FIG. 2 is a cross-sectional view illustrating an example of an over-tube provided in the medical system in FIG. 1.

FIG. 3A is a vertical sectional view illustrating a state where the over-tube in FIG. 2 extends in a straight line.

FIG. 3B is a vertical sectional view illustrating a state where the over-tube in FIG. 3A is bent.

FIG. 4 is a flowchart explaining the operation of the medical system in FIG. 1.

FIG. 5 is a partial vertical sectional view illustrating a state where all joints of the medical device protrude forward from the over-tube of the medical system in FIG. 1.

FIG. 6 is an overall configuration diagram schematically illustrating a modification of the medical system in FIG. 1.

FIG. 7 is a cross-sectional view illustrating a modification of the over-tube in FIG. 2.

FIG. 8 illustrates the arrangement of sensing wires in the over-tube in FIG. 7.

### DESCRIPTION OF EMBODIMENTS

A medical system 1 according to an embodiment of the present invention will be described below with reference to the drawings.

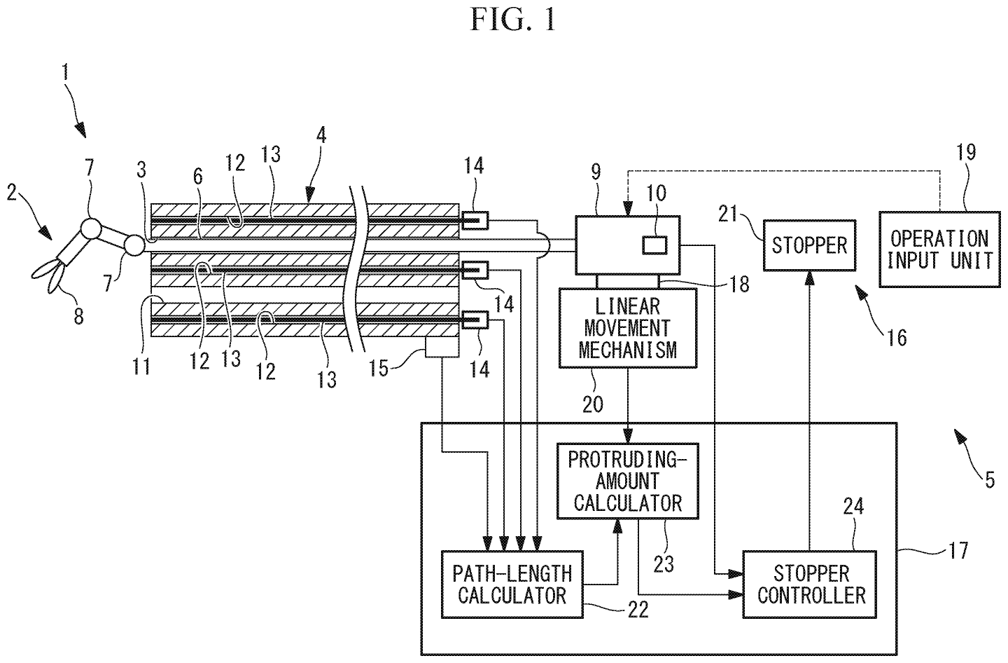

As shown in FIG. 1, the medical system 1 according to this embodiment includes a medical device 2, such as a medical manipulator, an over-tube 4 having a channel 3 through which the medical device 2 extends, and a main system unit 5 that connects the medical device 2 and the over-tube 4.

The medical device 2 includes an elongated section 6 having flexibility, at least one joint 7 and at least one surgical section 8 that are disposed at the distal end of the elongated section 6, and a driver 9 that is disposed at the proximal end of the elongated section 6 and that drives the joint 7 and the surgical section 8. The surgical section 8 is, for example, grasping forceps. The joint 7 and the surgical section 8 are connected to the driver 9 by a power transmitting member, such as a wire (not shown).

The medical device 2 includes a readable memory device 10 that stores information about the medical device 2. The information about the medical device 2 includes, for example, the length from the distal end of the medical device 2 to the joint 7 at the extreme proximal end in a state where the joint 7 of the medical device 2 extends in a straight line.

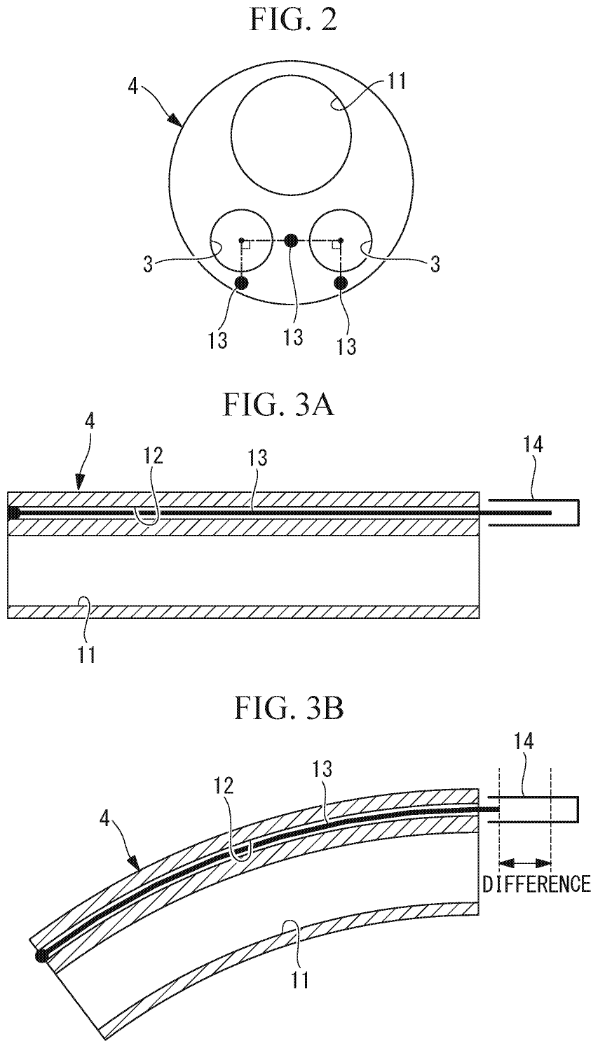

The over-tube 4 is an elongated tube having flexibility and, as shown in FIG. 2, includes an endoscope channel 11 through which an endoscope (not shown) extends and two channels 3 through which two medical devices 2 extend. The channel 11 and the channels 3 extend over the entire length in the longitudinal direction.

Furthermore, as shown in FIG. 1, the over-tube 4 includes three wire channels 12 extending parallel to the channels 3 and 11, three sensing wires (wires) 13 that are fitted in the respective wire channels 12 and whose distal ends alone are fixed to the distal end of the over-tube 4, and three sensors 14 that detect the protruding lengths of the sensing wires 13 from the proximal end of the over-tube 4. The sensors 14 may be of an electrostatic type, a magnetic type, or an optical type.

As shown in FIGS. 3A and 3B, when the over-tube 4 bends, the path length of the wire channel 12 disposed at the outer side of the bend increases, so that the sensing wire 13 fitted in the wire channel 12 is retracted into the wire channel 12, whereby the protruding length from the proximal end of the over-tube 4 decreases. The sensor 14 is capable of detecting this difference in the protruding length.

As shown in FIG. 2, in the cross section of the over-tube 4, the three sensing wires 13 are disposed at three respective locations that are decentered from the center of the over-tube 4 in the radial direction and that are not set on the same line extending through the center. In particular, in this embodiment, the positional relationship is set such that lines connecting the center of each of the channels 3 and 11 in the radial direction and two neighboring sensing wires 13 are orthogonal to each other.

The over-tube 4 also includes a readable memory device 15 that stores information about the over-tube 4. The information about the over-tube 4 includes, for example, the lengths of the channels 3 and 11 in a state where the over-tube 4 extends in a straight line.

The main system unit 5 includes a base (not shown) to which the proximal end of the over-tube 4 is secured, a movement mechanism 16 that is provided at the base, and a controller 17 that controls the movement mechanism 16. The driver 9 provided at the proximal end of the medical device 2 is attached to the movement mechanism 16, and the movement mechanism 16 moves the medical device 2 in the longitudinal direction relative to the over-tube 4.

The movement mechanism 16 includes a slider 18 to which the driver 9 is attached, an operation input unit 19 to be operated by an operator, a linear movement mechanism 20 that causes the slider 18 to move linearly in accordance with an operation amount input to the operation input unit 19, a position sensor (not shown) that detects the position of the slider 18, and a stopper 21 that can limit the retraction position of the slider 18. In this embodiment, the operation input unit 19 and the linear movement mechanism 20 are mechanically connected to each other.

The stopper 21 used may be, for example, any mechanism, such as an electromagnetic clutch, which limits the retraction position of the slider 18 by being driven in accordance with a command from the controller 17.

The controller 17 includes a reader (not shown) that reads the information in the memory devices 10 and 15 provided in the over-tube 4 and the medical device 2, a path-length calculator 22 that calculates the path lengths of the channels 3 based on the information read from the memory device 15 of the over-tube 4 and the protruding lengths of the three sensing wires 13 read by the three sensors 14, a protruding-amount calculator 23 that calculates the protruding amount of the medical device 2 from the distal end of the over-tube 4 based on the path lengths calculated by the path-length calculator 22 and the position of the slider 18 detected by the position sensor, and a stopper controller 24 that generates a signal for driving the stopper 21 based on the protruding amount calculated by the protruding-amount calculator 23 and the information read from the memory device 10 of the medical device 2.

The hardware configuration of the controller 17 includes at least one processor, at least one memory device, and a circuit that connects them.

The path-length calculator 22 calculates a path length in accordance with expression (1) indicated below.

Lc=√(Ls−X₁)²+(Ls−X₂)²) (1)

where

Lc indicates the path length of a channel 3 in a bent state,

Ls indicates the length of the channel 3 read from the memory device 15 in a state where the over-tube 4 extends in a straight line, and

X₁ and X₂ indicate differences between the protruding lengths detected by two sensors 14 adjacent to the channel 3 in a state where the over-tube 4 extends in a straight line and a state where the over-tube 4 is bent, respectively.

The protruding-amount calculator 23 calculates the protruding amount of the medical device 2 from the distal end of the over-tube 4 based on the position of the slider 18 detected by the position sensor and the path length calculated by the path-length calculator 22. For example, the driver 9 of the medical device 2 is connectable only at a position where the slider 18 is retracted maximally toward the proximal end. In a state where the over-tube 4 extends in a straight line, the distal end of the medical device 2 is set at the distal-end position of the over-tube 4 when the driver 9 is connected to the slider 18.

Accordingly, the protruding amount of the medical device 2 from the distal end of the over-tube 4 in a state where the over-tube 4 is bent is as follows:

Lx=Lp+Ls−Lc (2)

where

Lx indicates the protruding amount, and

Lp indicates the position of the slider 18.

Specifically, when the over-tube 4 bends and the path length Lc of the channel 3 increases, the protruding amount Lx decreases by the amount of increase (Lc−Ls).

Based on the protruding amount Lx and the length Lj, read from the memory device 10, from the distal end of the medical device 2 to the joint 7 at the extreme proximal end in a state where the joint 7 of the medical device 2 extends in a straight line, the stopper controller 24 sets the stopper position such that:

Lx≥Lj (3)

The operation of the medical system 1 according to this embodiment having the above-described configuration will be described below.

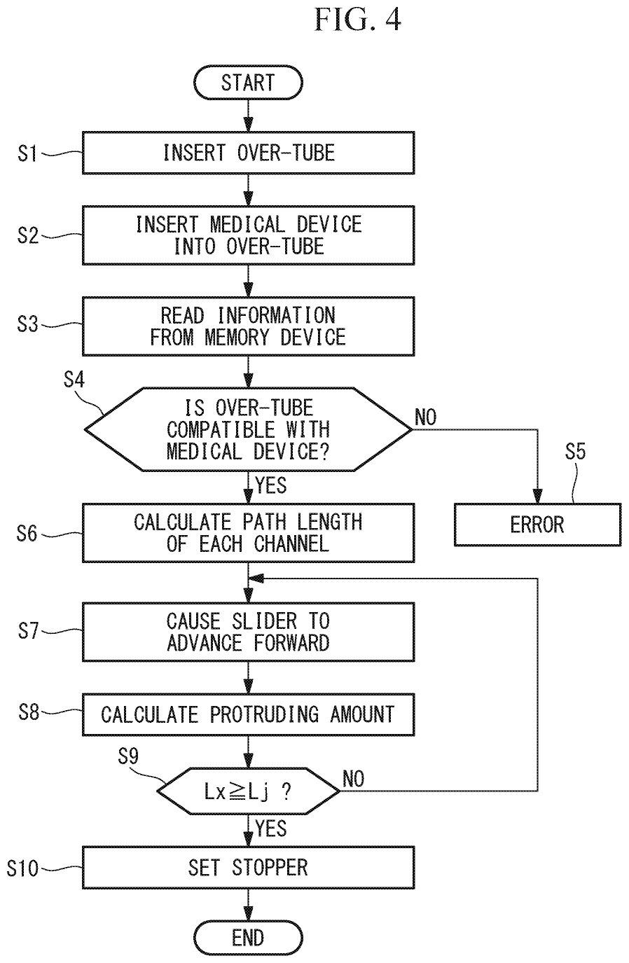

As shown in FIG. 4, in order to use the medical system 1 according to this embodiment, the over-tube 4 having an endoscope fitted in the endoscope channel 11 thereof is inserted into the body cavity of a patient while an endoscopic image is being checked (step S1). The over-tube 4 is inserted while being bent to conform to the shape of the body cavity, and is inserted to a position where an affected area is confirmed in the endoscopic image.

Subsequently, the medical device 2 is inserted into a channel 3 from the proximal end of the over-tube 4 disposed outside the patient's body (step S2). The medical device 2 is inserted while being bent to conform to the shape of the channel 3. In this state, the proximal end of the over-tube 4 is secured to the base of the main system unit 5, and the driver 9 at the proximal end of the medical device 2 is attached to the slider 18 of the movement mechanism 16 provided at the base. In this case, the slider 18 is retracted maximally toward the proximal end.

In this state, the distal end of the medical device 2 is disposed at a retracted position within the channel 3.

The information in the memory devices 10 and 15 provided in the over-tube 4 and the medical device 2 is read in this state (step S3). At this point, it is determined whether or not the over-tube 4 has a sufficient length (i.e., is compatible with) the medical device 2 (step S4). If the over-tube 4 does not have a sufficient length, an error notification is provided (step S5).

If the over-tube 4 has a sufficient length, the path-length calculator 22 subsequently calculates the path length of the channel 3 in accordance with expression (1) based on the protruding lengths of the sensing wires 13 detected by the sensors 14 (step S6).

When the operator operates the operation input unit 19 to cause the slider 18 to advance forward (step S7), the protruding-amount calculator 23 calculates the protruding amount of the medical device 2 from the distal end of the over-tube 4 in accordance with expression (2) based on the position of the slider 18 detected by the position sensor and the path length (step S8).

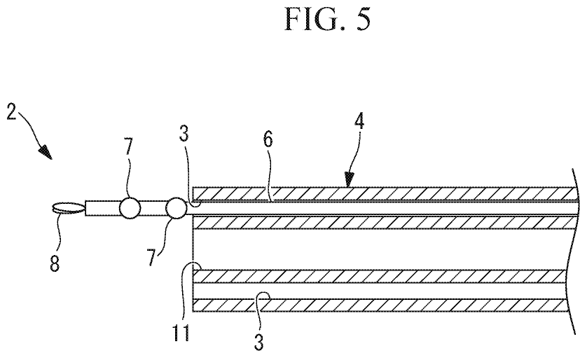

Then, it is determined whether or not the protruding amount satisfies expression (3) (step S9). If expression (3) is not satisfied, the process from step S7 is repeated. If expression (3) is satisfied, that is, when all joints 7 are disposed forward of the over-tube 4, as shown in FIG. 5, the stopper controller 24 sets the stopper 21 to restrict retraction of the slider 18 toward the proximal end from that position (step S10).

Specifically, expression (2) indicates the value of a protruding amount that allows the medical device 2 to protrude entirely to the joint 7 at the extreme proximal end from the distal end of the over-tube 4. Therefore, by setting the stopper 21 at a position where expression (2) is satisfied, the joint 7 of the medical device 2 can be reliably prevented from being retracted into the channel 3 even if the path length of the channel 3 increases as a result of bending of the over-tube 4.

When the surgery is completed and the medical device 2 is to be pulled out from the channel 3, the stopper 21 is released in accordance with another operation in a state where all of the joints 7 of the medical device 2 are extended in a straight line or in a state where the joints 7 are freely rotatable, so that the medical device 2 can be readily pulled out.

Moreover, if the operator desires to change the shape of the over-tube 4 while performing surgery using the medical device 2, the operator may perform the process from step S6 while temporarily keeping the medical device 2 accommodated in the channel 3.

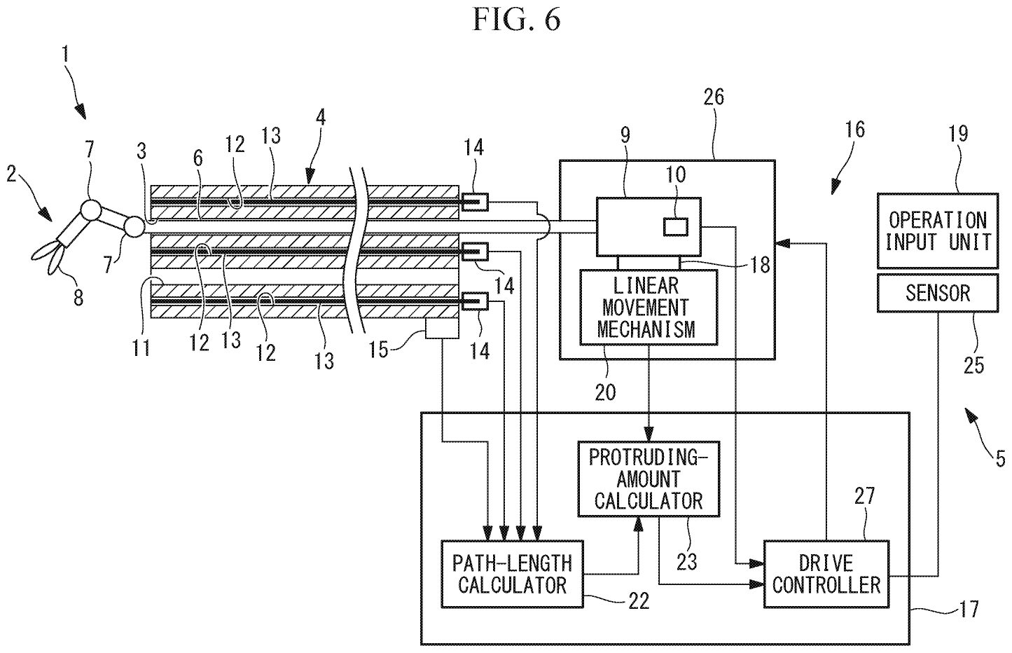

Furthermore, in this embodiment, the operation input unit 19 operated by the operator and the linear movement mechanism 20 are described as being mechanically connected to each other. Alternatively, as shown in FIG. 6, there may be provided an advance-retract driver 26 that uses an actuator, such as a motor, to drive the slider 18 based on an operation input from the operation input unit 19 and detected by a sensor 25. In this case, the stopper 21 and the stopper controller 24 may be replaced by a drive controller 27 that limits the driving of the actuator of the advance-retract driver 26 by using software.

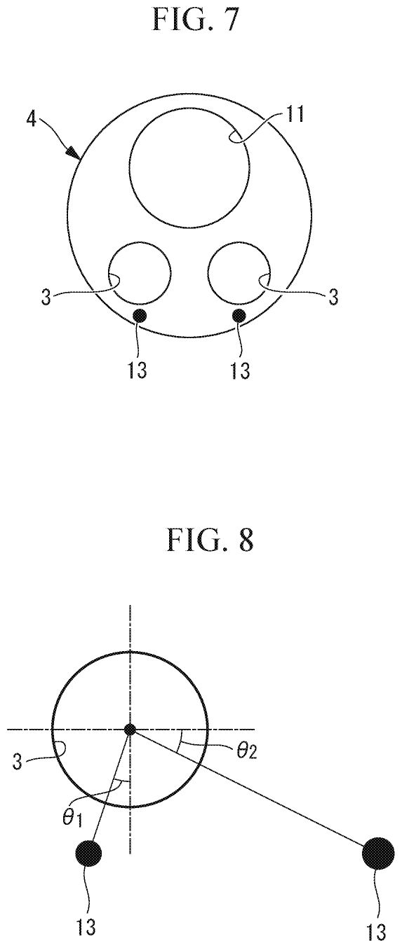

In this embodiment, there are three sensing wires 13 disposed for calculating the path lengths of two channels 3. Alternatively, two sensing wires 13 may be disposed for each channel 3, or two sensing wires 13 may be provided for two channels 3, as shown in FIG. 7. In this case, as shown in FIG. 8, in the cross section of the over-tube 4, the two sensing wires 13 may be disposed apart from each other on a line disposed parallel to and away from a line extending through the center.

In this case, the path lengths may each be calculated in accordance with expression (4) indicated below, in place of expression (1).

Lc=√(n×((Ls±X₁)cos θ₁))²+(n+((Ls±X₂)cos θ₂))²) (4)

where

n indicates a ratio of the bend radius of the over-tube 4 to the bend radius of the medical device 2, and

θ₁ and θ₂ indicate angles of lines connecting the sensing wires 13 and the center of the channel 3 relative to lines extending through the center of the channel 3 and orthogonally to each other.

If there is a single channel 3 for the medical device 2, only a single sensing wire 13 may be provided. In this case, if the over-tube 4 is bent about a line extending through the sensing wire 13 and the center of the over-tube 4, the path length is not detectable since the sensing wire 13 does not bend. Therefore, if the over-tube 4 does not bend in that direction, such an over-tube 4 may be used.

A notifier (not shown) that provides a notification when a path length is calculated by the path-length calculator 22 may be provided. The operator moves the medical device 2 in accordance with the reported path length, so that the joint 7 of the medical device 2 can be prevented from being retracted into the channel 3.

The above-described embodiment also leads to the following invention.

An aspect of the present invention provides a medical system including: an elongated medical device having flexibility; an elongated over-tube having flexibility and having a channel into which the medical device is inserted in a longitudinal direction; and a main system unit having a base to which a proximal end of the over-tube is secured, a movement mechanism that is provided at the base and to which a proximal end of the medical device is detachably attached, and a controller that controls the movement mechanism. The movement mechanism moves the medical device in the longitudinal direction relative to the over-tube. The over-tube is provided with a wire and a sensor. The wire is fixed to a distal end of the over-tube, extends parallel to the channel, and is supported in a relatively movable manner only in the longitudinal direction. The sensor detects a protruding length of the wire from the proximal end of the over-tube. The controller calculates a path length of the channel based on the protruding length of the wire detected by the sensor.

According to this aspect, the over-tube is inserted into the body cavity of a patient, and the proximal end of the over-tube, which is exposed to the outside of the patient's body, is secured to the base of the main system unit. When being inserted into the body cavity of the patient, the flexible over-tube bends in conformity to the shape of the body cavity. Accordingly, the channel provided in the over-tube and the wire extending parallel to the channel are deformed into the same shape as the over-tube.

As a result of the bent over-tube, the path length of the channel changes, and the wire fixed to the distal end of the over-tube and supported in a movable manner only in the longitudinal direction moves in the longitudinal direction relatively to the over-tube, thus resulting in a change in the protruding length from the proximal end of the over-tube. Since the protruding length of the wire is detected by the sensor and the positions of the channel and the wire in the radial direction are known, the path length of the channel can be readily and accurately calculated based on the protruding length when the over-tube extends in a straight line and the protruding length when the over-tube is bent.

Subsequently, the medical device is inserted into the channel from the proximal end of the over-tube, and the proximal end of the medical device exposed from the proximal end of the over-tube is attached to the movement mechanism provided at the base. Accordingly, the medical device can be retracted by the movement mechanism in accordance with the calculated path length of the channel.

In the above aspect, the wire may include two or more wires disposed apart from each other in a direction intersecting a radial direction of the over-tube.

Accordingly, even when the over-tube bends about an arbitrary axis extending through the center thereof in the radial direction and orthogonal to the longitudinal axis, the protruding length of at least one of the wires from the proximal end of the over-tube changes, so that the path length of the channel can be accurately calculated.

Furthermore, in the above aspect, the controller may control the movement mechanism based on the calculated path length.

Accordingly, the medical device can be disposed at a desired position in the longitudinal direction of the over-tube based on the accurately-calculated path length.

Furthermore, in the above aspect, the controller may control the movement mechanism such that a protruding amount of the medical device from the distal end of the over-tube is larger than or equal to a predetermined threshold value.

Accordingly, based on the accurately-calculated path length, the medical device can be disposed such that the medical device protrudes from the distal end of the over-tube by a protruding amount that is larger than or equal to the predetermined threshold value.

Furthermore, in the above aspect, the movement mechanism may include a stopper that is capable of limiting retraction of the medical device to a predetermined position, and the controller may control the stopper based on the calculated path length.

Accordingly, the movement of the medical device can be limited by the stopper such that the medical device is prevented from being retracted into the channel by more than or equal to a predetermined protruding amount from the distal end of the over-tube.

Furthermore, in the above aspect, the medical system may further include a notifier that provides a notification about the calculated path length.

Accordingly, the operator can adjust the movement amount of the medical device in accordance with the reported path length.

### REFERENCE SIGNS LIST

1 medical system
2 medical device
3 channel
4 over-tube
5 main system unit
13 sensing wire (wire)
14 sensor
16 movement mechanism
17 controller
21 stopper

1. A medical system comprising:
an elongated medical device having flexibility;
an elongated over-tube having flexibility and having a channel into which the medical device is inserted in a longitudinal direction;
a movement mechanism including a slider attached to a proximal end of the medical device, the movement mechanism being configured to move the medical device in the longitudinal direction relative to the over-tube;
a wire fixed to a distal end of the over-tube and extending parallel to the channel, the wire being supported in a movable manner only in the longitudinal direction relative to the over-tube;
a sensor that detects a protruding length of the wire from the proximal end of the over-tube; and
a controller that calculates a path length of the channel based on the protruding length of the wire detected by the sensor.

2. The medical system according to claim 1,
wherein the wire includes two or more wires disposed apart from each other in a direction intersecting a radial direction of the over-tube.

3. The medical system according to claim 1,
wherein the controller controls the movement mechanism based on the calculated path length.

4. The medical system according to claim 3,
wherein the controller controls the movement mechanism such that a protruding amount of the medical device from the distal end of the over-tube is larger than or equal to a predetermined threshold value.

5. The medical system according to claim 4,
wherein the movement mechanism includes a stopper that is configured to limit retraction of the slider to a predetermined position, and
wherein the controller controls the stopper based on the calculated path length.

6. The medical system according to claim 1, further comprising:
a notifier that provides a notification about the calculated path length.
